(19) United States
(12) Patent Application Publication
(10) Pub. No.: US 2022/0265214 A1
(43) Pub. Date: Aug. 25, 2022

(54) WEARABLE BIOSENSING DEVICE

(71) Applicant: Empatica Srl, Milano (IT)

(72) Inventors: Parth JARIWALA, Brookline, MA (US); Matteo LAI, San Sperate (IT); Simone TOGNETTI, Vimodrone (IT); Daniele RESNATI, Carate Brianza (IT); Nicolo BONACINA, Monza E Della Brianza (IT); Chris LOUGHNANE, Brookline, MA (US); Ivan CENCI, Verucchio (IT)

(21) Appl. No.: 17/743,739

(22) Filed: May 13, 2022

Related U.S. Application Data
(63) Continuation of application No. PCT/US2020/061125, filed on Nov. 18, 2020.
(60) Provisional application No. 62/937,046, filed on Nov. 18, 2019.

(51) Int. Cl. A61B 5/00; A61B 5/0205; A61B 5/28; A61B 5/291; A61B 5/296; A61B 5/024; A61B 5/1455; A61B 5/145

(52) U.S. Cl. CPC A61B 5/681; A61B 5/02055; A61B 5/28; A61B 5/291; A61B 5/296; A61B 5/02416; A61B 5/02438; A61B 5/02405; A61B 5/14552; A61B 5/7203; A61B 5/14539; A61B 5/14532; A61B 5/14546; A61B 2562/0219; A61B 2562/0271; A61B 2562/164; A61B 2562/166

(57) ABSTRACT

Embodiments described herein relate generally to a portable biometric monitoring device having a central pod, sensor electrodes, and a wearable band. The central pod can be removably coupled to the wearable band. The sensor electrodes can transfer data to an circuit on a printed circuit board (PCB) in the central pod. The circuit can be housed in the central pod and can be configured to process data transferred from the sensor electrodes. The central pod can be electrically coupled to the sensor electrodes via one or more conductive wires while the wearable band is coupled to the central pod. In some embodiments, the central pod can be electronically isolated from the sensor electrodes when the wearable band is not coupled to the central pod. In some embodiments, the one or more conductive wires are substantially encased in the wearable band.

### CROSS-REFERENCE TO RELATED APPLICATIONS

This application claims the benefit of U.S. Provisional Application No. 62/937,046, filed on Nov. 18, 2019, the entire disclosure of which is incorporated herein by reference in its entirety.

### TECHNICAL FIELD

Embodiments described herein relate generally to portable biometric monitoring devices.

### BACKGROUND

Embodiments described herein relate generally to a portable biometric monitoring device. Biometric monitoring devices include activity trackers, smartwatches and other monitoring devices. Biometric monitoring devices can aid in tracking fitness-related metrics such as distance walked or run, calorie consumption, heart rate, and other metrics. While biometric monitoring devices can track and share important biometric information, they often have to be removed for charging and/or for data downloads/uploads.

### SUMMARY

Embodiments described herein relate generally to a portable biometric monitoring device having a central pod, sensor electrodes, and a wearable band. In some embodiments, the sensor electrodes can include electrodermal activity (EDA) sensor electrodes, electromyography (EMG) sensor electrodes, electrocardiogram (EKG) sensor electrodes, electroencephalogram (EEG) sensor electrodes, microfluidic sensor electrodes, pH sensor electrodes, glucose sensor electrodes, DNA sensor electrodes, phosphate sensor electrodes or any combination thereof. The central pod can be removably coupled to the wearable band. The sensor electrodes can transfer data to a circuit on a printed circuit board (PCB) in the central pod. The circuit can be housed in the central pod and can be configured to process data transferred from the sensor electrodes. The central pod can be electrically coupled to the sensor electrodes via one or more conductive wires while the wearable band is coupled to the central pod. In some embodiments, the central pod can be electronically isolated from the sensor electrodes when the wearable band is not coupled to the central pod. In some embodiments, the one or more conductive wires are substantially encased in the wearable band. In some embodiments, the portable biometric monitoring device includes a photoplethysmogram (PPG) sensor having a PPG sensor surface and the PPG sensor surface is configured to be coupled to either the ventral side or the dorsal side of a user's wrist. In some embodiments, the biometric monitoring device can include a plurality of pins located on a surface different from the PPG sensor surface. In some embodiments, the plurality of pins can be used for transferring electrical energy (e.g., via an electric current) to the central pod (i.e., charging) and/or for transferring data. The plurality of pins can be located on a surface orthogonal or substantially orthogonal to the PPG sensor surface. In some embodiments, the biometric monitoring device can include a secondary device configured to be removably coupled to the plurality of pins.

### BRIEF DESCRIPTION OF THE DRAWINGS

FIG. 1 is a schematic illustration of a biometric monitoring device, according to an embodiment.

FIGS. 2A-2B are perspective views of a biometric monitoring device, according to an embodiment.

FIGS. 3A-3B are perspective views of a central pod of a biometric monitoring device, according to an embodiment.

FIGS. 4A-4D are perspective views of a wearable band, according to an embodiment.

FIGS. 5A-5B are perspective views of a secondary device, according to an embodiment.

FIGS. 6A-6B are perspective views of a biometric monitoring device, according to an embodiment.

FIG. 7 depicts a view of a central pod of a biometric monitoring device according to an embodiment.

### DETAILED DESCRIPTION

Embodiments described herein relate generally to portable biometric monitoring devices that include a central pod, sensor electrodes, and a wearable band such that the portable biometric monitoring device can be wrist-worn. A biometric monitoring device is an apparatus that converts a biometric trait of an individual (e.g., pulse, blood pressure) into electrical signals. Biometric monitoring devices often include semiconductor devices that process data from an individual's physical characteristics using a series of algorithms. Biometric monitoring devices are often worn to track an individual's fitness metrics, but can also be used to monitor health conditions, such as, for example, high blood pressure, and for early detection of conditions and/or diseases, such as, for example, respiratory diseases including COVID-19. In addition to biometric traits, biometric monitoring devices can also be configured to track an individual's activity, such as distance walked or run, and level of intensity of physical activity. Additionally, biometric monitoring devices often include photoplethysmogram (PPG) sensing devices

In some embodiments, the sensor electrodes can include EDA sensor electrodes, EMG sensor electrodes, EKG sensor electrodes, EEG sensor electrodes, microfluidic sensor electrodes, pH sensor electrodes, glucose sensor electrodes, DNA sensor electrodes, phosphate sensor electrodes, or any combination thereof. In some embodiments, portable biometric monitoring devices described herein can be disposed around a user's wrist, chest, shoulder, waist, thigh, calf, knee, ankle, foot, toe, hand, neck, finger, forearm, bicep, head, or any other body part where measurements are desired.

Portable biometric monitoring devices often include an accelerometer and a gyroscope, in addition to the PPG module. These devices can therefore continuously sense the movements of a human body on a 3-axis accelerometer. Movement data is recorded while the device is worn and it enables the device to trace if the user is walking, running, or standing still. In addition to the movement data, PPG data can be used to measure pulse, blood pressure, and other cardiovascular parameters. Movement data and PPG data can then be stored for further processing. The movement data and PPG data are generally provided to a software program housed in the device, or the movement data is sent to an external machine (e.g., smartphone, computer, etc.) for further processing. Taking into account the user's personal details (e.g., height, weight, etc.) the software can determine what is implied by the data it receives and develop reasonable statistics. The software can categorize movements into different activities based on rate of movement and heart rate (e.g., walking, running, bicycling) and then generate more information based on these details. This information can be in the form of a user's average steps per day, resting heart rate, or general physical fitness level. The information can be provided to the user via an application either in a computer, in a smartphone, or in the portable biometric monitoring device itself.

PPG is an optically obtained data set that can be used to detect blood volume changes in a user's microvascular bed of tissue. A PPG is often obtained by using a series of light emitting diodes (LEDs) that illuminate the user's skin and measure changes in light absorption. PPG modules and the collection of PPG data is described in U.S. Pat. No. 10,285,602, entitled, “Device, system and method for detection and processing of heartbeat signals,” (“the '602 patent”), the disclosure of which is incorporated herein by reference in its entirety.

Furthermore, some embodiments described herein relate to portable biometric monitoring devices that include an EDA sensor. EDA is the property of the human body that causes continuous variation in the electrical characteristics of the skin. EDA and apparatus for collecting EDA are described in U.S. Patent Publication No. 2014/0316229, entitled, “Apparatus for electrodermal activity measurement with current compensation,” (“the '229 publication”) the disclosure of which is incorporated herein by reference in its entirety.

Biometric monitoring devices often require removal of the device while charging. In other words, the user cannot wear the device 100% of the time. This can be particularly problematic for users and/or third parties (e.g., remote patient monitoring clinicians) monitoring health conditions. This problem can be overcome via the development of a biometric monitoring device that can be charged in place while being worn.

As used in this specification, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

The term “substantially” when used in connection with “cylindrical,” “linear,” and/or other geometric relationships is intended to convey that the structure so defined is nominally cylindrical, linear or the like. As one example, a portion of a support member that is described as being “substantially linear” is intended to convey that, although linearity of the portion is desirable, some non-linearity can occur in a “substantially linear” portion. Such non-linearity can result from manufacturing tolerances, or other practical considerations (such as, for example, the pressure or force applied to the support member). Thus, a geometric construction modified by the term “substantially” includes such geometric properties within a tolerance of plus or minus 5% of the stated geometric construction. For example, a “substantially linear” portion is a portion that defines an axis or center line that is within plus or minus 5% of being linear.

As used herein, the term “set” and “plurality” can refer to multiple features or a singular feature with multiple parts.

As used herein, the term “about” and “approximately” generally mean plus or minus 10% of the value stated, e.g., about 250 μm would include 225 μm to 275 μm, about 1,000 μm would include 900 μm to 1,100 μm.

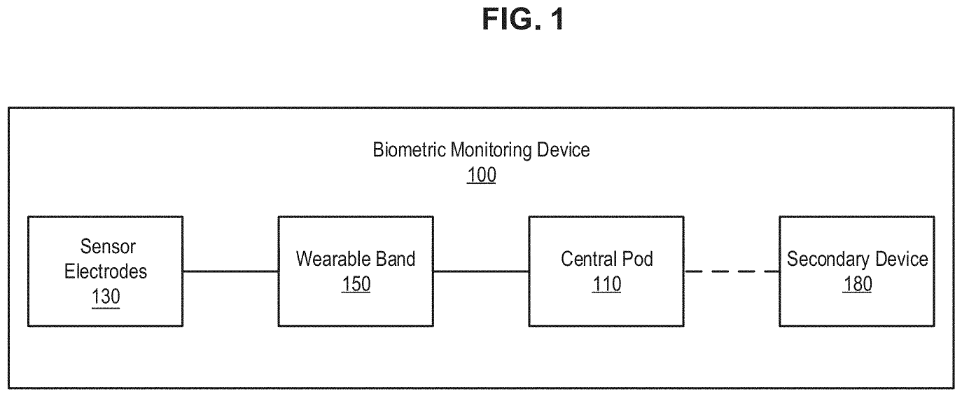

FIG. 1 is a schematic illustration of a biometric monitoring device 100, according to an embodiment. The biometric monitoring device 100 includes a central pod 110, sensor electrodes 130, and a wearable band 150. The central pod 110 can be removably coupled to the wearable band 150. In some embodiments, the biometric monitoring device 100 can include a secondary device 180 that is configured to be removably coupled to the central pod 110. In some embodiments, the central pod 110, the sensor electrodes 130, the wearable band 150, and the secondary device 180 can be highly water resistant, such that the user can wear the biometric monitoring device 100 while showering or swimming.

In some embodiments, the central pod 110 can include a PPG module, gyroscope, Bluetooth antenna, and/or an accelerometer housed inside the central pod 110. In some embodiments, the biometric monitoring device 100 can include a temperature sensor. In some embodiments, the temperature sensor can be appended to and/or housed inside the central pod 110. In some embodiments, the PPG module can have any of the features described in the '602 patent. In some embodiments, the PPG module can measure oxygen saturation (SpO₂). In some embodiments, the PPG module can measure heart rate variability (HRV). In some embodiments, the PPG module can remove noise from signals input to the PPG module (e.g., raw data measured by the PPG module). The PPG module can illuminate the user's skin through a transparent PPG module surface. In some embodiments, the central pod 110 can include a charging port (not shown), wherein the charging port includes a plurality of charging pins (not shown). In some embodiments, the charging port can be on a different surface than the PPG module surface. In some embodiments, the charging port can be on a surface oriented approximately orthogonal to the PPG module surface. If the charging port is on a different surface from the PPG module surface, the secondary device 180 can be attached to the charging port to charge the biometric monitoring device 100 while the biometric monitoring device 100 is being worn by the user. This can allow the biometric monitoring device to be worn constantly. In some embodiments, the charging pins can be configured to allow the transfer of electrical energy (e.g., via an electric current) and/or data. In some embodiments, the central pod 110 can include a display screen and one or more buttons that the user can press to adjust settings and/or what information is presented on the display screen.

The sensor electrodes 130 are electronically coupled to the central pod 110. In some embodiments, the sensor electrodes 130 can include EDA sensor electrodes, EMG sensor electrodes, EKG sensor electrodes, EEG sensor electrodes, microfluidic sensor electrodes, pH sensor electrodes, glucose sensor electrodes, DNA sensor electrodes, phosphate sensor electrodes, or any combination thereof. In some embodiments, the sensor electrodes 130 can be configured to make physical contact with the ventral side of the user's wrist. In some embodiments, the sensor electrodes 130 can have any of the features described in the '229 publication. In some embodiments, the sensor electrodes 130 can include two or more electrodes. In some embodiments, the sensor electrodes 130 can be electronically coupled to the central pod 110 through conducive elements or channels such as, for example, conductive wires (not shown). In some embodiments, the conductive wires can be encased in and/or integrated into the wearable band 150. In other words, the conductive wires can run through an interior of the wearable band 150, e.g., such that the conductive wires are isolated or substantially isolated from contact with the user's skin and the atmosphere. In some embodiments, the sensor electrodes 130 can be coupled to the central pod 110 via a flexible circuit including one or more conductive paths. The flexible circuit can be integrated into, encased within, and/or otherwise supported by the wearable band 150. In some embodiments, the sensor electrodes 130 can be at least partially encased in the wearable band 150. For example, the sensor electrodes 130 can be disposed within the wearable band 150 such that a portion of the sensor electrodes 130 is covered by wearable band 150 and isolated or insulated from external signals. In some embodiments, the sensor electrodes 130 can be configured to measure EDA. In some embodiments, the biometric monitoring device 100 can include additional electrodes (not shown). In some embodiments, the additional electrodes can be configured to collect electrocardiogram (EKG), peripheral capillary oxygen saturation (SpO₂), or other biometric data.

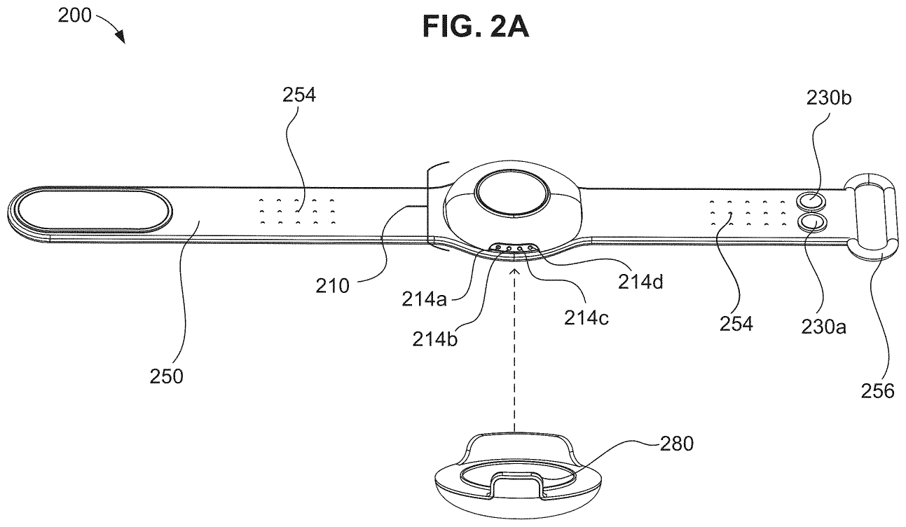

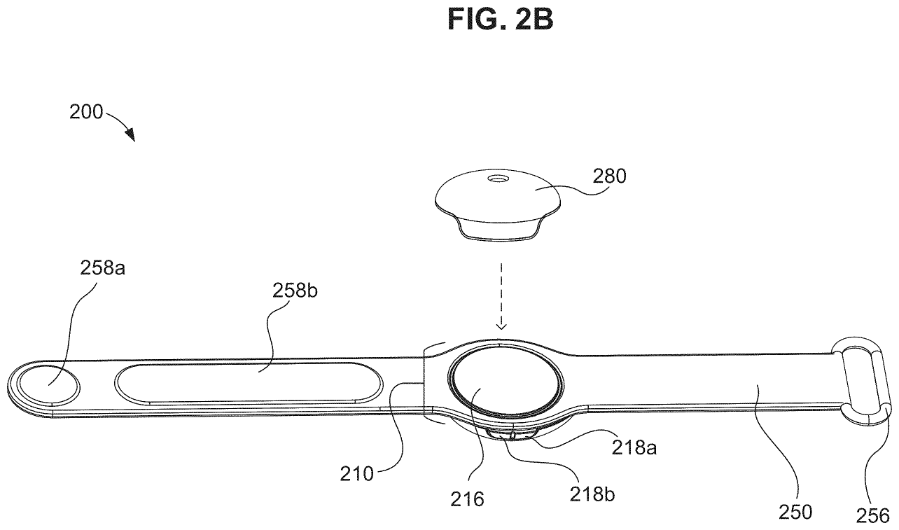

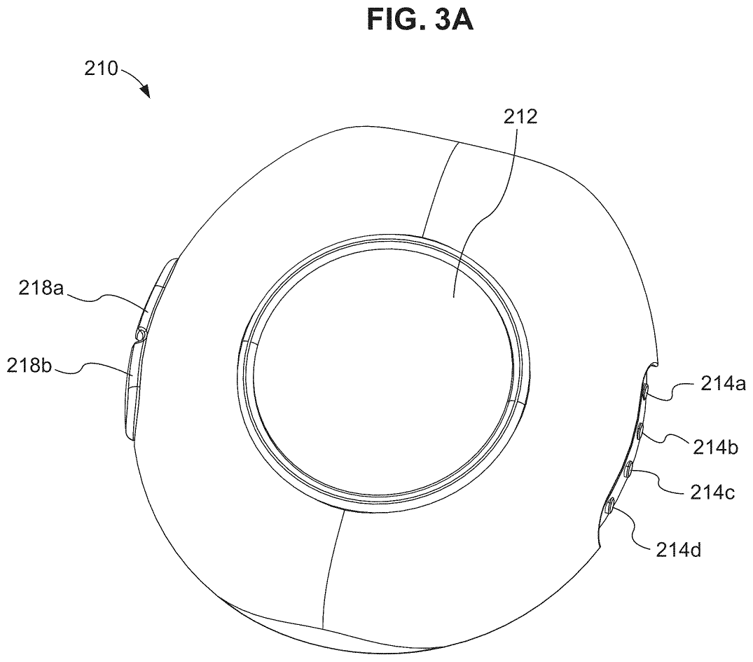

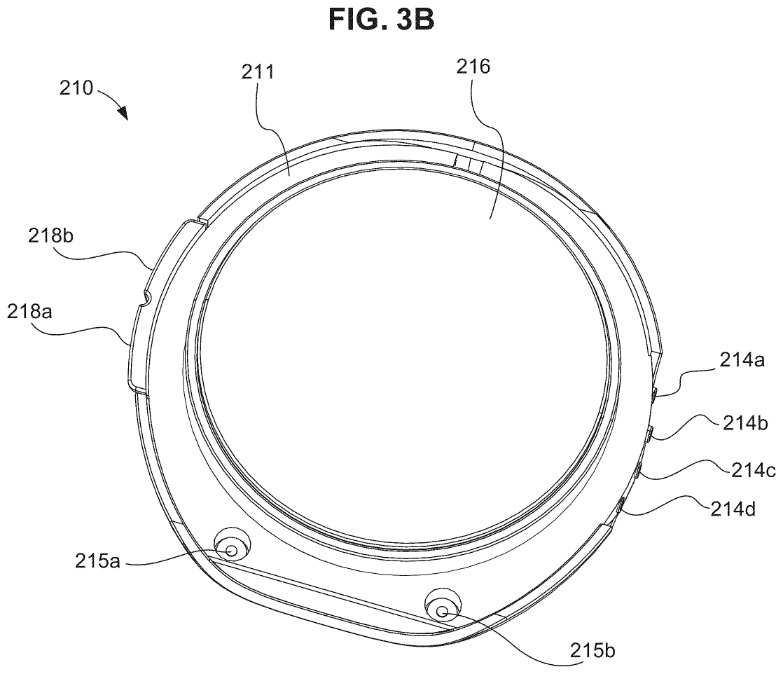

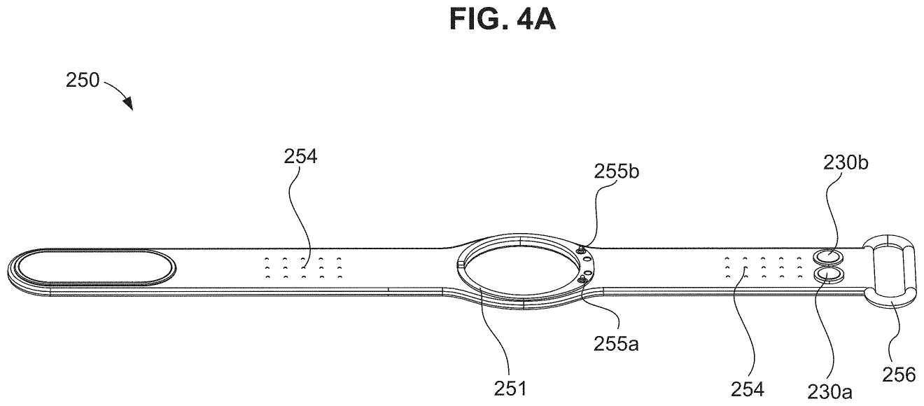

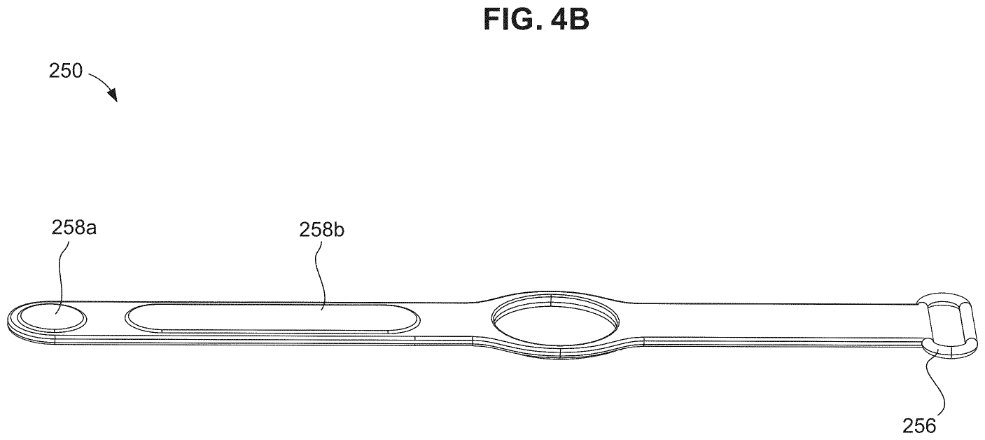

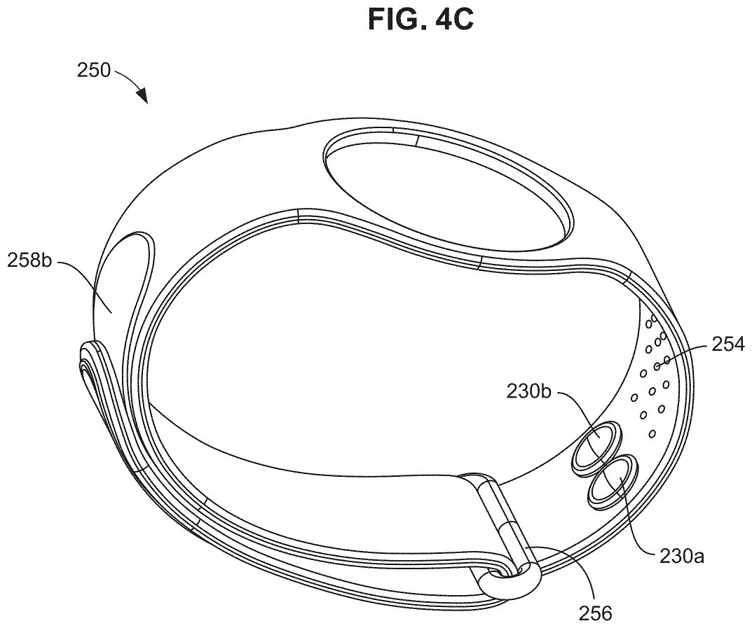

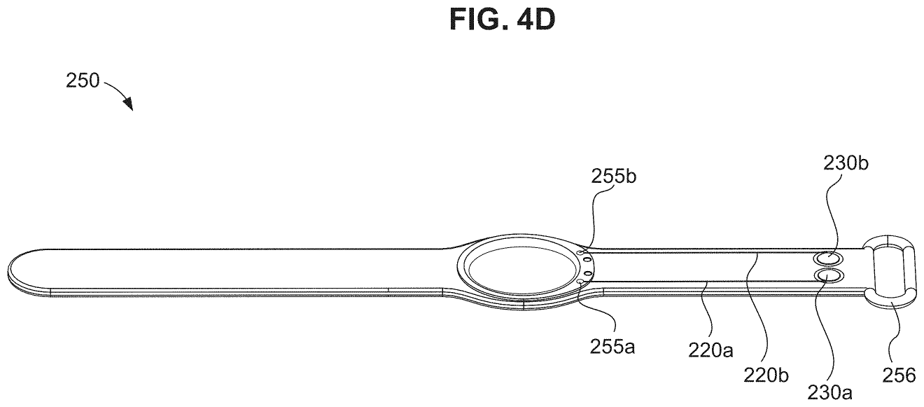

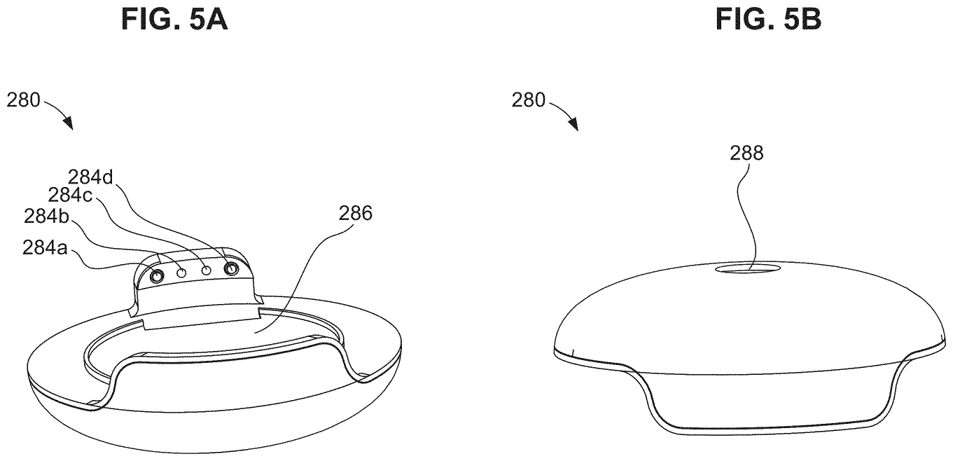

In some embodiments, the wearable band 150 can be configured to maintain or hold the central pod 110 and/or sensor electrodes 130 against a skin of the user. In some embodiments, the wearable band 150 can be configured to fit around a user's wrist, leg, and/or other appendage. In some embodiments, the wearable band 150 can be made from or include material that is forms high friction or resistance against skin (e.g., a high friction material), such that the wearable band 150 can reduce or prevent movement of the central pod 110 and/or sensor electrodes 130 when the wearable band 150 is worn on the user. In some embodiments, the wearable band 150 can include slip-resistant protuberances, as further described with reference to FIGS. 2A-5B. In some embodiments, the wearable band 150 can be made from or include an insulating or non-conductive material, e.g., such that the wearable band 150 can be configured to isolate one or more conductive writes and/or sensor electrodes 130 from one another. In some embodiments, the wearable band 150 can be configured to support and/or partially encase the sensor electrodes 130 and/or the conductive wires coupled to the sensor electrodes 130. In some embodiments, the wearable band 150 can be made from or include a sterilizable material, a medical grade material (e.g., a biocompatible material), a stretchable material, a polymer, a plastic, a silicone, or any combination thereof.

In some embodiments, the central pod 110 and the wearable band 150 can be removably coupled via a magnetic coupling. The magnetic coupling between the central pod 110 and the wearable band 150 can aid in the ease of cleaning each component. In some embodiments, the sensor electrodes 130 can be electronically coupled to the central pod 110 when the wearable band 150 is coupled to the central pod 110. In some embodiments, the sensor electrodes 130 can be electronically isolated from the central pod 110 while the wearable band 150 is removed from to the central pod 110. In some embodiments, the wearable band 150 can be adjustable, such that the fit of the biometric monitoring device 100 is configured to the user's preference. In some embodiments, the wearable band 150 can be adjustable, such that the electrodes of the sensor electrodes 130 are in the desired location relative to the ventral side of the user's wrist. In some embodiments, the wearable band 150 can include slip-resistant protuberances. In some embodiments, the wearable band 150 can include a PPG module. In some embodiments, the PPG module can be encased in the wearable band 150. In some embodiments, the PPG module can be affixed to the wearable band 150.

The secondary device 180 can have one or more functions. In some embodiments, the secondary device 180 can be removably coupled to the central pod 110. In some embodiments, the secondary device 180 can be removably coupled to the central pod 110 via a magnetic coupling. In some embodiments, the secondary device 180 can include a charging apparatus. The secondary device 180 can include a battery or other energy storage device that can be charged using a conventional cable or dock, and then that energy storage device discharges when connected to the central pod 110, thereby charging the central pod 110. This removable secondary device 180 can allow for the biometric sensing device 100 to be worn continuously, such that it can be charged without being removed from the user's wrist. This functionality can be particularly important for a user with a medical condition, for which constant monitoring is desired (e.g., epilepsy). In some embodiments, the secondary device 180 can include software similar to software found in the central pod 110. In some embodiments, the secondary device 180 can be configured to extract physiological data from the central pod 110. In some embodiments, the secondary device 180 can include Wi-Fi, Bluetooth, and/or cellular communication. In some embodiments, the secondary device 180 can have a secondary antenna or range extender to improve the connectivity range of the central pod 110. In some embodiments, the secondary device 180 can be configured to upload data the secondary device 180 extracts from the central pod 110 to an external location, such as a cell phone, a computer, and/or a server (i.e., “the cloud”). In some embodiments, the secondary device 180 can collect additional physiological data that the central pod 110 does not collect. In some embodiments, the secondary device 180 can collect EKG data, EMG data, EDA data, EEG data, microfluidic data, pH data, glucose sensor electrodes, DNA sensor electrodes, phosphate sensor electrodes. In some embodiments, the secondary device 180 can collect similar physiological data to the physiological data that the central pod 110 collects. This can be for backup or redundancy purposes. This can also act as a means of improving the quality of the data collected by the central pod 110 (e.g., removal of motion artifact data).

In some embodiments, the secondary device 180 can collect contextual data, including but not limited to sound data, ambient light data, and/or weather data. In some embodiments, the secondary device 180 can transfer any of the data it collects to the central pod 110 via direct wired transfer or through wireless communication. In some embodiments, the secondary device 180 can transfer data to an external device (e.g., computer, cell phone, server, etc.), where the data can be processed and/or analyzed and then the data can be sent to the central pod 110. The central pod 110 can use the processed data to enhance the performance of its algorithms. In some embodiments, the secondary device 180 can have a data collection sensor configured to communicate data to the central pod 110. Data communicated to the central pod 110 can then be transferred to an external device. In some embodiments, the secondary device 180 can be larger than the central pod 110, such that it has more space for data transmission ports (e.g., USB ports) or charging ports.

In some embodiments, the secondary device 180 can include LEDs, an E-ink display, and/or a matrix LED display. The LEDs and/or E-ink display can be used to communicate any values relating to its current state, including but not limited to any information communicated via the display unit on the central pod 110. In some embodiments, the secondary device 180 can include one or more buttons, capacitive-touch screen, and/or a resistive touch screen, which can be used to query any status value of the secondary device 180 and/or the central pod 110. The one or more buttons, capacitive-touch screen, and/or resistive touch screen can also be used to change any settings on the central pod 110 and/or the secondary device 180. In some embodiments, the secondary device 180 can have gesture recognition capabilities, such that the secondary device 180 can learn to associate various gestures or motions from the user with the user's desire to query any status value of the secondary device 180 and/or the central pod 110. The aforementioned various gestures can also be used to change any settings on the central pod 110 and/or the secondary device 180. In some embodiments, the secondary device 180 can assume the function of the central pod 110 if the central pod 110 has no remaining battery life, is having communication problems, or is otherwise not performing all of its desired functions. In some embodiments, the central pod 110 and the secondary device 180 can each include a magnetic sensor, such that the central pod 110 and the secondary device 180 can detect each other's presence.

In some embodiments, the biometric monitoring device 100 can include components such as a communications module, a processing module, etc., such as, for example, those described in U.S. Patent Application Publication No. 2014/0316229, titled “Apparatus for electrodermal activity measurement with current compensation,” filed Mar. 17, 2014, U.S. Patent Application Publication No. 2015/0327787, titled “Device, system and method for detection and processing of heartbeat signals,” filed Jul. 24, 2015, and U.S. Pat. No. 8,140,143, filed Apr. 16, 2009, titled “Washable wearable biosensor,” the contents of each of which are incorporated herein by reference.

FIGS. 2A-5B show multiple perspective views of a biometric monitoring device 200 and components of the biometric monitoring device 200, according to various embodiments. The biometric monitoring device 200 can include component(s) that are structurally and/or functionally similar to those of other biometric monitoring devices described herein (e.g., biometric monitoring device 100). As shown, the biometric monitoring device 200 includes a central pod 210, conductive wires 220a, 220b (collectively referred to as conductive wires 220), sensor electrodes 230a, 230b (collectively referred to as sensor electrodes 230), a wearable band 250, and a secondary device 280. The central pod 210 includes a coupling surface 211, a PPG module surface 212, pins 214a, 214b, 214c, and 214d (collectively referred to as pins 214), pogo pin contacts 215a, 215b (collectively referred to as pogo pin contacts 215), a display screen 216, and buttons 218a, 218b (collectively referred to as buttons 218). As shown, the wearable band 250 includes a band coupling surface 251, slip-resistant protuberances 254, pogo pins 255a, 255b (collectively referred to as pogo pins 255), a buckle 256, and Velcro surfaces 258a, 258b (collectively referred to as Velcro surfaces 258). As shown, the secondary device 280 includes pin contacts 284a, 284b, 284c, 284c (collectively referred to as pin contacts 284), a bottom surface 286, and a top surface 288.

In some embodiments, the central pod 210 can have any of the same capabilities as the central pod 110 described above with reference to FIG. 1. As shown, the central pod 210 can be removably coupled to the wearable band 250. The coupling between the central pod 210 and the wearable band 250 can be achieved by joining central pod coupling surface 211 and the wearable band coupling surface 251. In some embodiments, the central pod coupling surface 211 and the wearable band coupling surface 251 can be joined magnetically. While the central pod 210 is coupled to the wearable band 250, the pogo pins 255 make physical contact with the pogo pin contacts 215.

The central pod 210 is electronically connected to the sensor electrodes 230 via the conductive wires 220 when the central pod 210 is coupled to the wearable band 250. The conductive wires 220 make physical contact with the pogo pins 255 and the sensor electrodes 230. In some embodiments, the conductive wires 220 can be encased in the wearable band 250. In other words, the conductive wires 220 can join the pogo pins 255 and the sensor electrodes 230 via channels on the interior of the wearable band 250. In some embodiments, the conductive wires 220 can be composed of copper, copper-covered steel, high strength copper alloys, aluminum, or any other conductive materials. In some embodiments, the conductive wires 220 can be coupled to the pogo pins 255 and/or the sensor electrodes 230 via soldering, welding, brazing, or any other joining process. In some embodiments, the conductive wires 220 can be coated in an insulating material to enhance their electronic isolation from the atmosphere and the user's skin. In some embodiments, the conductive wires 220 can be coated in an insulating material. In some embodiments, the conductive wires 220 can be coated in Teflon. In some embodiments, the pogo pins 255 can be composed of copper, copper-covered steel, high strength copper alloys, aluminum, or any other conductive materials. In some embodiments, the pogo pins 255 can be plated in a material resistant to corrosion, such as gold or silver. In some embodiments, the pogo pins 255 can be composed of a material resistant to corrosion, such as gold or silver. In some embodiments, the pogo pin contacts 215 can be composed of copper, copper-covered steel, high strength copper alloys, aluminum, or any other conductive materials. In some embodiments, the pogo pin contacts 215 can be plated in a material resistant to corrosion, such as gold or silver. In some embodiments, the pogo pin contacts 215 can be composed of a material resistant to corrosion, such as gold or silver. As shown, the biometric monitoring device 200 includes two of each of the sensor electrodes 230, conductive wires 220, pogo pins 255, and pogo pin contacts 215. In some embodiments, the biometric monitoring device 200 can include three, four, five, six, seven, eight, or more of each of the sensor electrodes 230, conductive wires 220, pogo pins 255, and pogo pin contacts 215.

As shown, the central pod 210 includes a PPG module. In some embodiments, the PPG module can function via LEDs that illuminate the user's skin by radiating through the PPG module surface 212. While in use, the PPG module surface 212 can be coupled to the user's skin.

As shown, the secondary device 280 can be removably coupled to the central pod 210. In some embodiments, the secondary device 280 can have any of the same capabilities as the secondary device 180 described above, with reference to FIG. 1. The central pod pins 214 can be in physical contact with the pin contacts 284. In some embodiments, the central pod pins 214 can be composed of copper, copper-covered steel, high strength copper alloys, aluminum, or any other conductive materials. In some embodiments, the central pod pins 214 can be plated in a material resistant to corrosion, such as gold or silver. In some embodiments, the central pod pins 214 can be composed of a material resistant to corrosion, such as gold or silver. In some embodiments, the pin contacts 284 can be composed of copper, copper-covered steel, high strength copper alloys, aluminum, or any other conductive materials. In some embodiments, the pin contacts 284 can be plated in a material resistant to corrosion, such as gold or silver. In some embodiments, the pin contacts 284 can be composed of a material resistant to corrosion, such as gold or silver. As shown, the biometric monitoring device 200 includes four of each of the central pod pins 214 and the pin contacts 284. In some embodiments, the biometric monitoring device can include three, five, six, seven, eight, or more of each of the central pod pins 214 and the pin contacts 284. In some embodiments, charging can occur via the contact between the central pod pins 214 and the pin contacts 284. In some embodiments, data can be shared via the contact between the central pod pins 214 and the pin contacts 284. As shown, the central pod pins 214 are on a different surface than the PPG module surface 212. As described above with reference to FIG. 1, this can allow the user to attach a charging device (i.e., the secondary device 280) and charge the biometric monitoring device 200 while the PPG module and the sensor electrodes 230 are still collecting data. As shown, the central pod pins 214 are oriented approximately orthogonal to the PPG module surface 212.

The display screen 216 and buttons 218 can have properties similar to the properties of the one or more buttons and display screen described above with reference to FIG. 1. As shown, the biometric monitoring device 200 includes two buttons. In some embodiments, the biometric monitoring device 200 can include one, three, four, five, six, seven, eight, or more buttons. In some embodiments, the display screen 216 can be a capacitive-touch screen, and/or a resistive touch screen.

Additional components of the wearable band 250 include the slip-resistant protuberances 254, the buckle 256, and the Velcro surfaces 258. The slip-resistant protuberances 254 can keep the wearable band 250 from rotating around the user's wrist while being worn. This can be important for keeping the sensor electrodes 230 in the proper position on the ventral side of the user's wrist, and also for reducing motion artifacts in data collected by the sensor electrodes 230. The side of the wearable band 250 distal to the buckle 256 can be threaded through the buckle 256, adjusted to a desired fit, and fastened via the Velcro surfaces 258. As shown, the wearable band 250 is fastened via Velcro. In some embodiments, the wearable band 250 can be fastened via a prong and holes or any other fastening mechanism.

In some embodiments, the secondary device 280 can have any of the same capabilities as the secondary device 180 described above with reference to FIG. 1. Additional components of the secondary device 280 include the bottom surface 286 and the top surface 288. In some embodiments, when the secondary device 280 is coupled to the central pod 210, the bottom surface 286 can be coupled to the display screen 216. In some embodiments, the top surface 288 can include an additional screen that can display information while the secondary device 280 is coupled to the central pod 210. In some embodiments, the top surface 288 can include a button. In some embodiments, the top surface 288 can include a capacitive-touch screen, and/or a resistive touch screen.

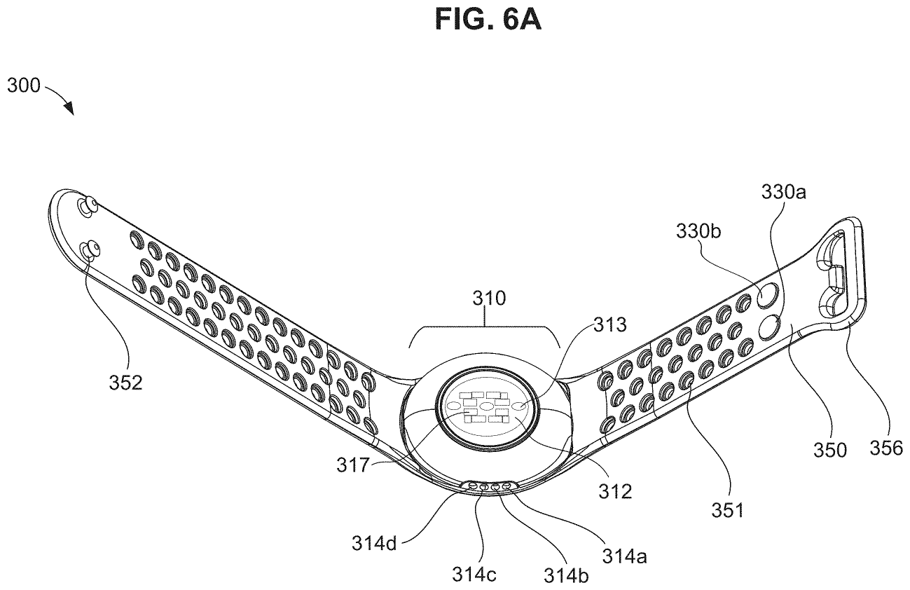

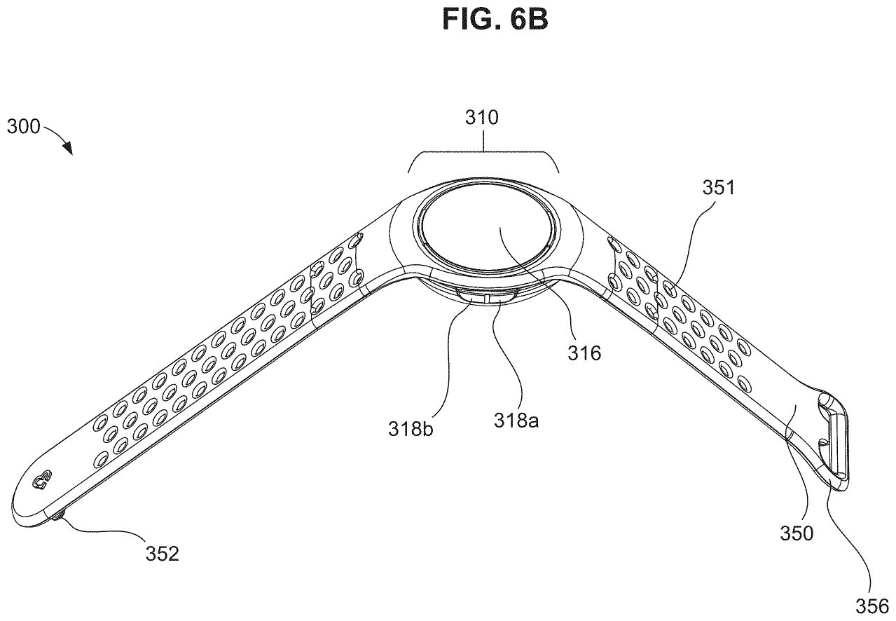

FIGS. 6A-6B are perspective views of a biometric monitoring device 300, according to an embodiment. FIG. 6A shows an interior side of the monitoring device 300 configured to contact the user's body while FIG. 6B shows a side of the monitoring device 300 configured to be displayed. The biometric monitoring device 300 can include component(s) that are structurally and/or functionally similar to those of other biometric monitoring devices described herein (e.g., biometric monitoring device 100, 200).

As shown, the biometric monitoring device 300 includes a central pod 310, sensor electrodes 330a, 330b (collectively referred to as sensor electrodes 330), and a wearable band 350. The central pod 310 includes a PPG module surface 312, LEDs 313, photodiodes (PDs) 317, pins 314a, 314b, 314c, and 314d (collectively referred to as pins 314), a display screen 316, and buttons 318a, 318b (collectively referred to as buttons 318). As shown, the wearable band 350 includes adjustment holes 351, adjustment prongs 352, and a buckle 356. In some embodiments, the biometric monitoring device 300 can include conductive wires or conductive channels (e.g., printed on a flexible printed circuit board) (not shown) and/or a secondary device (not shown). In some embodiments, conductive wires and the secondary device can be the same or substantially similar to the conductive wires 220 secondary device 280, as described above with reference to FIGS. 2A-5B. In some embodiments, the conductive wires can couple the central pod 310 to the sensor electrodes 330. In some embodiments, the central pod 310, the PPG module surface 312, the pins 314, the display screen 316, the buttons 318, the sensor electrodes 330, the wearable band 350, the adjustment holes 351, the adjustment pegs 352, and the buckle 356 can be the same or substantially similar to the central pod 210, the PPG module surface 212, the pins 214, the display screen 216, the buttons 218, the sensor electrodes 230, the wearable band 250, the adjustment holes 251, the adjustment pegs 252, and the buckle 256, as described above with reference to FIGS. 2A-5B. Thus, certain aspects of the central pod 310, the PPG module surface 312, the pins 314, the display screen 316, the buttons 318, the sensor electrodes 330, the wearable band 350, the adjustment holes 351, the adjustment pegs 352, and the buckle 356 are not described in greater detail herein.

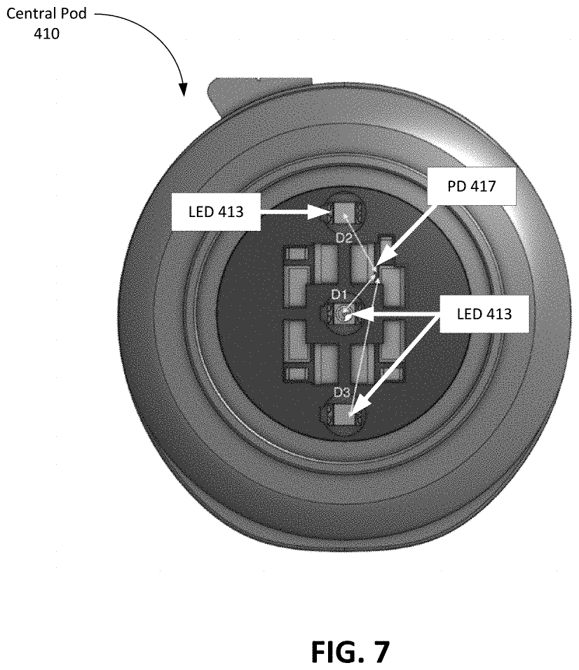

In some embodiments, the central pod 310 can be removable from the wearable band 350. In some embodiments, the conductive wires can run through the wearable band 350 and contact the sensor electrodes 330. In some embodiments, the sensor electrodes 330 can be configured to contact the ventral side of the user's wrist. In some embodiments, the sensor electrodes 330 can be configured to contact the dorsal side of the user's wrist. In some embodiments, the LEDs 313 and/or the PDs 317 can be configured to have certain operational parameters or properties (e.g., intensity, wavelength or color of light, etc.) and/or be specifically placed based on the measurement to be conducted (e.g., EKG, EDA). In some embodiments, the LEDs 313 and the PDs 317 can be optically separated by a light barrier to avoid crosstalk between the LEDs and the PDs 317. In some embodiments, the central pod 310 can include at least 1, at least 2, at least 3, at least 4, at least 5, at least 6, at least 7, at least 8, at least 9, or at least 10 LEDs 313. In an embodiment, the central pod 310 can include three LEDs, including, for example, LEDs that emit different wavelengths of length (e.g., green, red, infrared). In some embodiments, each LED 313 or subsets of LEDs 313 can be independently driven by circuitry through different channels. In some embodiments, one or more of the LEDs 313 can be covered by a lens (e.g., a special lens) to increase light emission efficiency. In some embodiments, the central pod 310 can include at least 1, at least 2, at least 3, at least 4, at least 5, at least 6, at least 7, at least 8, at least 9, or at least 10 PDs 317. In some embodiments, the signals generated by the PDs 317 can be acquired independently by the circuitry through different channels. In some embodiments, each of the PDs 317 can be disposed at a non-symmetrical distance from the LEDs 313 to optimize the chance of getting a quality signal for a greater portion of potential users. FIG. 7 depicts an embodiment of a central pod 410 with an example placement of PDs 417 at non-symmetrical distances from LEDs 413. With such placement, multiple configurations can be selected for light to travel through different volumes of skin (e.g., distances D1, D2, D3).

In some embodiments, the wearable band 350 can be composed of a sterilizable material, a medical grade material, a stretchable material, a polymer, a plastic, a silicone, or any combination thereof. As shown, the side of the wearable strap 350 that includes the adjustment pegs 352 can be pulled through the buckle 356 and the adjustment pegs 352 can be inserted into the adjustment holes 351 at a desired size. In some embodiments, the buckle 356 can be shaped such that the opening created by the buckle 356 is larger where the adjustment pegs 352 move through the buckle 356. For example, if the wearable strap 350 includes two adjustment pegs 352, the buckle 356 can include an opening with two enlarged portions to accommodate for the adjustment pegs 352 to be inserted into the buckle 356. Such a design can ease adjustment of the size or tightness of the wearable band 350.

The biometric monitoring devices (e.g., 100, 200, 300) disclosed herein can include a processor, a memory, and an input/output device (e.g., a display, a communications module, etc.). While not specifically described above with the biometric monitoring devices, the central pod of the biometric monitoring devices can include a display that provides certain information to a user, e.g., information representative of or summarizing measured physiological data (e.g., EDA data, heartrate, SpO₂, etc.), information representative of or summarizing contextual data (e.g., weather data, time and date, location, etc.), remaining battery life, wireless connectivity status, reminders, alerts, etc. The display can be disposed on a surface of the central pod that is opposite from a surface including one or more sensors (e.g., the PPG module surface). In some embodiments, the biometric monitoring devices (e.g., 100, 200, 300) can be wirelessly coupled to one or more external devices, e.g., a user device such as a mobile phone, tablet, laptop, computer, etc. In some embodiments, the biometric monitoring devices (e.g., 100, 200, 300) can include a user input interface, including a touch screen, button, etc.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1-34. (canceled)

35. A portable biometric monitoring device, comprising:
a central pod;
a wearable band removably coupleable to the central pod, the wearable band configured to be worn around a bodily structure of a subject;
a plurality of sensor electrodes mounted to the wearable band such that the wearable band is configured to maintain the plurality of sensor electrodes in contact with a skin of the subject when the wearable band is worn around the bodily structure, the plurality of sensor electrodes configured to measure physiological data of the subject; and
one or more conductive channels configured to provide electronic contact between the central pod and the plurality of sensor electrodes when the wearable band is coupled to the central pod, the one or more conductive channels encased within and extending along at least a portion of the wearable band.

36. The portable biometric monitoring device of claim 35, wherein the plurality of sensor electrodes includes at least one of: an EDA sensor electrode, an electromyography (EMG) sensor electrode, an electrocardiogram (EKG) sensor electrode, an electroencephalogram (EEG) sensor electrode, a microfluidic sensor electrode, a pH sensor electrode, a glucose sensor electrode, a DNA sensor electrode, or a phosphate sensor electrode.

37. The portable biometric monitoring device of claim 35, wherein the central pod is electrically isolated from the plurality of sensor electrodes when the wearable band is not coupled to the central pod.

38. The portable biometric monitoring device of claim 35, wherein the bodily structure of the subject is a wrist of the subject,
the central pod including a photoplethysmogram (PPG) module and a PPG module surface, the PPG module surface configured to be in contact with either a ventral side or a dorsal side of the wrist when the wearable band is worn around the wrist.

39. The portable biometric monitoring device of claim 38, wherein the PPG module is configured to measure at least one of oxygen saturation (spO₂), heart rate, or heart rate variability (HRV).

40. The portable biometric monitoring device of claim 38, wherein the PPG module is configured to remove noise from signals measured by the PPG module.

41. The portable biometric monitoring device of claim 38, wherein the central pod includes a plurality of pins, the plurality of pins configured to transfer at least one of electrical energy or data between the central pod and a secondary device coupled to the central pod.

42. The portable biometric monitoring device of claim 41, wherein the plurality of pins are located on a surface approximately orthogonal to the PPG module surface.

43. The portable biometric monitoring device of claim 41, further comprising the secondary device, the secondary device removably coupleable to the plurality of pins, the secondary device configured to transfer at least one of the electrical energy or the data to the central pod.

44. The portable biometric monitoring device of claim 35, wherein the central pod includes a temperature sensor.

45. The portable biometric monitoring device of claim 35, wherein the central pod includes an accelerometer.

46. The portable biometric monitoring device of claim 35, wherein the wearable band is composed of at least one of: a biocompatible material, a stretchable material, a sterilizable material, an insulating material, or a high friction material.

47. The portable biometric monitoring device of claim 35, wherein the wearable band is composed of silicone.

48. The portable biometric monitoring device of claim 35, wherein at least one of the plurality of sensor electrodes is at least partially encased in the wearable band.

49. The portable biometric monitoring device of claim 35, wherein the one or more conductive channels are disposed on a flexible printed circuit board.

50. The portable biometric monitoring device of claim 35, wherein the central pod is configured to receive signals representative of the physiological data measured by the plurality of sensor electrodes and to transmit the signals to a compute device separate from the portable biometric monitoring device.

51. A portable biometric monitoring device, comprising:
a central pod;
a wearable band removably coupleable to the central pod, the wearable band configured to be worn around a bodily structure of a subject;
a plurality of sensor electrodes mounted to the wearable band such that the wearable band is configured to maintain the plurality of sensor electrodes in contact with a skin of the subject when the wearable band is worn around the bodily structure, the plurality of sensor electrodes being electrically coupled to the central pod when the wearable band is coupled to the central pod; and
a secondary device removably coupleable to the central pod, the secondary device configured to transfer at least one of electrical energy or data between the central pod and the secondary device.

52. The portable biometric monitoring device of claim 51, wherein the plurality of sensor electrodes includes at least one of: an EDA sensor electrode, an electromyography (EMG) sensor electrode, an electrocardiogram (EKG) sensor electrode, an electroencephalogram (EEG) sensor electrode, a microfluidic sensor electrode, a pH sensor electrode, a glucose sensor electrode, a DNA sensor electrode, or a phosphate sensor electrode.

53. The portable biometric monitoring device of claim 51, further comprising:
one or more conductive channels that electrically couple the plurality of sensor electrodes to the central pod when the wearable band is coupled to the central pod.

54. The portable biometric monitoring device of claim 51, wherein the central pod is electrically isolated from the plurality of sensor electrodes when the wearable band is not coupled to the central pod.
